(19) United States
(12) Patent Application Publication
(10) Pub. No.: US 2018/0250221 A1
(43) Pub. Date: Sep. 6, 2018

(54) GARDENIA EXTRACT FOR COLOURING THE SKIN

(71) Applicant: PIERRE FABRE MEDICAMENT, Boulogne-billancourt (FR)

(72) Inventors: Hélène DUPLAN, Auzeville Tolosan (FR); Christel FIORINI-PUYBARET, Toulouse (FR); Carine JACQUES-JAMIN, Tournefeuille (FR); Philippe JOULIA, Villenouvelle (FR); Laurent SUBRA, Montgaillard (FR)

(73) Assignee: PIERRE FABRE MEDICAMENT, Boulogne-billancourt (FR)

(21) Appl. No.: 15/758,043

(22) PCT Filed: Sep. 8, 2016
(86) PCT No.: PCT/EP2016/071157

§ 371 Date: Mar. 7, 2018

(30) Foreign Application Priority Data
Sep. 9, 2015 (FR) 1558358

(51) Int. Cl. A61K 8/9789; A61Q 19/04; A61K 8/60

(52) U.S. Cl. CPC A61K 8/9789; A61K 2800/5922; A61K 8/60; A61Q 19/04

(57) ABSTRACT

The present invention relates to the use of a cosmetic composition intended for colouring the skin, said cosmetic composition including, as an active principle, an aqueous extract of gardenia.

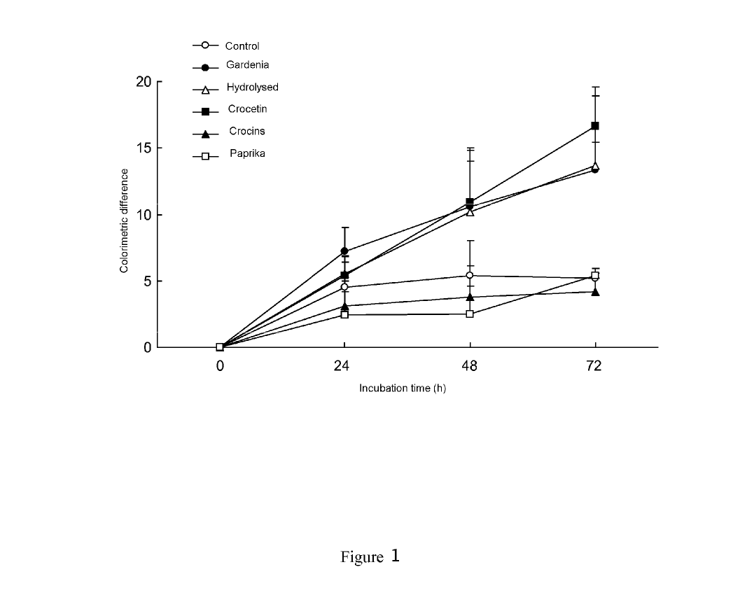

The present invention concerns the use of a Gardenia extract for colouring the skin.

Human skin colour, also called complexion, has a continuous gradation from white to an almost-black dark brown, sometimes with rose or copper tones. Skin colour is a striking individual characteristic which is often deemed an ethnic or socio-economic marker. The amount and nature of the pigments contained in the skin, as well as the distribution thereof, are the main criteria which determine skin colour. Genetics plays a key role in determining complexion, but sun exposure does too, as most humans tan under the effect of ultraviolet radiation. Both the absorption of certain medicinal products and diseases can cause hyperpigmentation or hypopigmentation. The surface distribution of three pigments is responsible for skin colour. Carotene is a pigment whose colour varies from yellow to orange. It accumulates in the cornea layer and the adipose cells of the hypodermis and promotes melanin synthesis. It is present in certain plants such as carrots. The second pigment is haemoglobin; when the skin is well oxygenated, haemoglobin gives light skin its rosy colour. Inadequate oxygenation gives it a bluish colour. Melanin is the most important pigment for skin colour. Melanin level determines skin colour and provides a correspondingly strong protection against ultraviolet rays. The epidermis and the hair of the head and body are coloured by melanin, which is found in the deepest layer of the epidermis, the basal layer, and which is produced by large cells, the melanocytes. Melanin is synthesized in the melanosomes from an amino acid, tyrosine. Tyrosinase is a key enzyme in melanin synthesis. It is the number and size of the melanosomes which varies the melanin level from one skin type to another. Melanin absorbs ultraviolet and infrared rays and captures the highly-toxic free radicals responsible for most of the sun's harmful effects.

Self-tanner is a cosmetic product used to obtain a temporary tan similar to that obtained by sun tanning, but without exposure to the sun. It must be applied to the body in order to colour the epidermis. Self-tanner contains dihydroxyacetone, a chemical agent which colours the skin. There is thus a great demand to develop new, easy-to-use products which effectively colour the skin by means of a simple oral dose, for example.

Unexpectedly, the inventors have found that a Gardenia extract via the oral and local route could be useful for colouring the skin.

Originating in China and Japan, Gardenia jasminoides J. Ellis is an evergreen shrub of the Rubiaceae family, generally about 1 to 2 m in height. This plant is cultivated in hot and humid tropical countries. The dark-green leaves are opposite, elliptic to oval-oblong, 5 to 10 cm in length and 2 to 7.5 cm in width, cuneiform at the base and acute or acuminate at the apex with a short petiole and with stipules fused in pairs. The white to ivory flowers are bell-shaped, 3 to 4 cm in size, solitary, terminal, sessile and very fragrant. The fruit is a tough, 5-sided berry, 1 to 1.5 cm in length, ovoid or ellipsoid, crowned with a persistent calyx, yellow to red at maturity and containing many seeds. The ripe fruit, harvested in the autumn and dried, is registered in the Chinese pharmacopoeia.

Chinese medicine prescribes Gardenia fruit in various preparations: dried fruit (Zhi-zi), fried fruit (Chaozhi-zi) or charred fruit (Jiaozhi-zi). It is prescribed for internal use as an antipyretic against bacillary dysentery, lung and urinary infections and hepatitis, or as a haemostatic in nasal haemorrhages caused by fever; and for external use to treat injuries, eye inflammation, contusions, wounds and boils. In Japanese Kampo medicine, the fruit is used to treat pain, lung disease and jaundice. These traditional uses can be explained by the pharmacological properties of the fruit, which is haemostatic, anti-inflammatory, stimulatory, cholagogic, emetic and diuretic.

The other parts of Gardenia jasminoides have many virtues. The antipyretic leaves are crushed in Malaysia to make poultices for treating migraines and pulmonary inflammation. The antipyretic and tonic bark is used for fever, dysentery and stomach pain. In India, the root is used for dyspepsia and nervous disorders. The emollient flowers are useful for treating ophthalmia, gonorrhoea and vaginal inflammation. The seeds are used externally in paste form to treat jaundice, rheumatism and diverticulosis.

In addition to these medicinal uses, the fruit are also used to dye foodstuffs or textiles yellow to orange owing to their high content of crocins, which are pigments identical to those of saffron.

The principal compounds of the fruit are:

iridoids, represented chiefly by geniposide and gardenoside. The other iridoids present as well as the following compounds in smaller quantities: 6″-O-trans-sinapoylgenipin gentiobioside, 6″-O-trans-p-coumaroylgenipin gentiobioside, 6″-O-trans-cinnamoylgenipin gentiobioside, 6″-O-trans-p-coumaroylgeniposide, 6′-O-trans-p-coumaroylgeniposide acid, 10-O-succinoylgeniposide, 6′-O-acetylgeniposide, Gardenal-I, Gardenal-II, Gardenal-III, 6-β-hydroxygeniposide,6-α-hydroxygeniposide, 6-α-methoxygeniposide, feretoside, genipin-1-β-gentiobioside, shanzhiside, and lamalbidic and picrocrocinic acids.

carotenoids such as croceic acid, crocetin and the crocins, glycosylated derivatives of crocetin. Crocin 1 (crocetin gentiobioside), crocin 2 (crocetin gentiobioside glucoside) and crocin 3 (crocetin glucoside) can be distinguished,

flavonoids: gardenin, quercetin, quercetin-3-rutinoside, quercetin-3-O-glucopyranoside, isoquercitrin, corymbosin, umuhengerin, nicotiflorin.

caffeylquinic derivatives (3-caffeoylquinic acid, 4-caffeoylquinic acid), 3,4-dicaffeoylquinic acid, 3,5-dicaffeoylquinic acid, 4,5-dicaffeoylquinic acid, ethyl 5-O-caffeoylquinate, methyl 5-O-caffeoyl 3-O-sinapoylquinate, ethyl 5-O-caffeoyl 3-O-sinapoylquinate,methyl 5-O-caffeoyl 4-O-sinapoylquinate, ethyl 5-O-caffeoyl 4-O-sinapoylquinate, methyl 3,5-di-O-caffeoyl-4-O-(3-hydroxy-3-methyl) glutaroylquinate, 3-O-caffeoyl-4-O-sinapoylquinic acid,methyl 3-O-caffeoyl-4-O-sinapoylquinate, methyl 3-O-caffeoyl-5-O-sinapoylquinate, 3,4-di-O caffeoyl-5-O-(3-hydroxy-methyl) glutaroylquinic acid, 3,5-di-O-caffeoyl-4-O-(3-hydroxymethyl) glutaroylquinic acid.

phenolic acids such as chlorogenic, caffeic and 3,4-dihydroxy-benzoic acids.

lignans: gardenianan A, syringaresinol, pinoresinol, syringaresinol-4-O-β-D-glucopyranoside, lariciresinol, alangilignoside D, lyoniresinol, lyoniresinol-9-O-β-D-glucopyranoside, balanophonin, glycosmisic acid, ficusal and ceplignan.

sugars (mannitol).

The present invention concerns the use of a cosmetic composition for colouring the skin, said cosmetic composition comprising as active ingredient an aqueous extract of Gardenia.

Preferably, the aqueous extract of Gardenia comes from the species Gardenia jasminoides.

According to an embodiment of the invention, the aqueous extract of Gardenia used to colour the skin contains a mass fraction of crocins of 0.1 to 10%, preferentially of 1 to 5%.

The composition according to the invention can be formulated for administration to human beings. The compositions according to the invention can be administered via the oral, sublingual, subcutaneous, intramuscular, intravenous, transdermal, topical or intranasal route. Advantageously, the composition according to the present invention is for administration via the topical route. Even more advantageously, the composition according to the present invention is for administration via the oral route.

In the latter case, the aqueous extract of Gardenia can be administered in unit dosage forms, mixed with conventional pharmaceutical carriers, to human beings. The suitable unit dosage forms comprise oral forms such as tablets, capsules, powders, granules and oral solutions or suspensions, sublingual and buccal dosage forms, subcutaneous or transdermal, topical, intramuscular, intravenous or intranasal dosage forms.

When a solid composition is prepared in tablet form, the principal active ingredient is mixed with a pharmaceutical carrier such as gelatin, starch, lactose, magnesium stearate, talc, gum arabic, silica or the like. The tablets can be coated with sucrose or other suitable materials or they can be treated so that they have a prolonged or delayed activity and that they continuously release a predetermined amount of active ingredient.

A preparation in capsules is obtained by mixing the active ingredient with a diluent and by pouring the mixture obtained into soft or hard capsules.

A preparation in syrup or elixir form can contain the active ingredient together with a sweetener, an antiseptic, as well as a flavouring agent and a suitable dye.

In a particular embodiment of the invention, the composition comprises, per unit dose, from 10 mg to 1 g of Gardenia extract, preferentially from 20 mg to 500 mg, advantageously from 50 mg to 250 mg and more preferentially from 100 mg to 200 mg;

In a particular embodiment of the invention, the composition comprises, per unit dose, from 0.2 mg to 20 mg of crocins, preferentially from 1 mg to 5 mg and more preferentially from 2 mg to 4 mg.

According to an embodiment of the invention, the Gardenia extract contains a mass fraction of crocins of 0.1 to 10%, preferentially of 0.5 to 8%, preferably of 1 to 5%, even more preferentially of 1 to 3%.

According to an embodiment of the invention, the Gardenia extract contains a mass fraction of crocetin of 0.01 to 5%, preferentially of 0.03 to 3%, even more preferentially of 0.1 to 1%.

According to a particularly advantageous embodiment of the invention, the cosmetic composition further comprises Paprika and/or another source of carotenoids derived from plants, microorganisms or microalgae, preferentially an extract of paprika (Capsicum annuum); astaxanthin-rich Haematoccus pluvialis; lutein- and zeaxanthin-rich African marigold (Tagetes erecta); tomato, a source of lycopene; Dunaliella salina, Xanthophyllomyces dendrorhous and Blakeslea trispora, sources of carotenes.

The present invention also concerns a cosmetic treatment method for colouring the skin, consisting in the administration of a cosmetic composition comprising an aqueous extract of Gardenia according to the invention.

Finally, the present invention concerns a process for extracting Gardenia fruit.

The Gardenia fruit are extracted with a solvent consisting of water or of a mixture of water and an organic solvent, an alcohol such as ethanol or an ester such as acetone. The extraction can be carried out on whole fruit or on fruit coarsely ground beforehand. Enzymes, such as pectinases, can be added in order to improve the extraction and/or the filtration thereof by fluidifying the extraction juices. The extraction can be carried out by a conventional method known to those skilled in the art, in a reactor, by ultrasound or by microwaves at a temperature of 20° C. to 100° C. depending on whether enzymes are present. The extraction can be carried out at atmospheric pressure or under pressure with subcritical water.

After solid-liquid separation, the filtrate is concentrated on water, sterilized then dried as it is or on a drying aid, such as maltodextrin or silica. Drying is carried out at temperatures below 80° C. in order to avoid degradation of the crocin molecules, which are unstable at higher temperatures. It can be carried out by techniques known to those skilled in the art, for instance by microwaves or spray drying.

The following examples illustrate the invention without limiting the scope thereof.

### EXAMPLE 1

### Extraction Process

Whole Gardenia fruit are extracted at 90° C. with water for 2 hours. After solid/liquid separation, pectinases are added. The filtrate is concentrated on water q.s. 40% dry extract, sterilized then dried with microwaves.

The dry aqueous extract obtained is a red powder. The yield is about 25%. The crocin contents are 0.1 to 10%, the average crocin content is of the order of 2%.

### EXAMPLE 2

### Pharmacological Effects on a Human Skin Explant Model

Owing to the lack of cell and animal pharmacological models, the inventors developed an ex vivo model in order to overcome the difficulties related to absorption of the active agents. Indeed, there is no absorption of crocin without intestinal hydrolysis first and low assimilation of crocetin and of these derivatives after passage.

### Protocol

The objective of this study was to evaluate the colouring effect of various extracts applied to human skin explants under survival conditions over 72 hours. Maintenance under survival conditions is carried out in 6-well culture plates containing culture medium. These culture plates are placed in an incubator at 37° C. with 5% CO₂. The studies were carried out on four donors.

The colouring of each medium containing active agents or control is evaluated with the C-Cube Dermoscope (Clinical Edition) from Pixience SAS. The evaluation concerns the maximum colouring which can be obtained, i.e., the 100% level on the colour mapping scale relating to each active agent. The colouring of each explant is measured using the C-Cube Dermoscope (Clinical Edition). First, the explant is placed in a solution of medium with no active agent in order to rinse the explant of the active agent molecules present on the explant surface and which have not penetrated the skin. Samples tested:
Control: culture medium
Paprika oleoresin (10% carotenoids)
Aqueous extract of Gardenia (4% crocins)
Aqueous extract of Gardenia hydrolysed chemically (2.7% crocetin)
Crocins (100%)
Crocetin (87%), obtained by chemical hydrolysis of crocins.

The culture medium selected is a Dulbecco's Modified Eagle Medium (DMEM), supplemented with glutamine, antifungals and antibiotics, described in the study by Jacques et al. 2010 (Toxicol. In Vitro 24(5), 1426-34).

Photographs of the skin explants, and the colorimetric differences illustrated in the single appended figure, showed the high colouring strength of crocetin, of Gardenia and of hydrolysed Gardenia.

Under these conditions, the colouring strength of Paprika and of crocins is inferior to those of crocetin, Gardenia and hydrolysed Gardenia.

These studies provide the proof of concept for colouring explants using the Gardenia extract, but also using the hydrolysed extract of Gardenia and using crocetin. These results were obtained on four donors, at various times and with different experimenters, thus validating the reproducibility of these results.

### EXAMPLE 3

### Bioavailability of Crocins

Few studies have been carried out to study the pharmacokinetic properties of crocins. The study of example 2 indeed showed the colouring strength of crocins but this result is obtained on human skin explants. However, the study carried out by Xi et al. in 2007 (Xi et al., Phytomedicine 14; 633-636, 2007) showed that crocins have a bioavailability of at least 20% in rats, which suggests even better results in humans. Page 2 of 3

1-7. (canceled)

8. A cosmetic treatment method for colouring the skin consisting in the administration of a cosmetic composition comprising as active ingredient an aqueous extract of Gardenia.

9. The cosmetic treatment method of claim 1 wherein said extract comes from the species Gardenia jasminoides.

10. The cosmetic treatment method of claim 1 wherein said extract contains a mass fraction of crocins of 0.1 to 10%, preferentially of 1 to 5%.

11. The cosmetic treatment method of claim 1 wherein said composition is administered via the oral route.

12. The cosmetic treatment method of claim 1 wherein said composition further comprises paprika and/or β-carotene.

13. The cosmetic treatment method of claim 1 wherein the aqueous extract of Gardenia is obtained by:
extraction from whole fruit in the presence of pectinases,
solid-liquid separation,
sterilization of the filtrate, and
drying at temperatures below 80° C.
